(19) United States
(12) Patent Application Publication
(10) Pub. No.: US 2025/0302783 A1
(43) Pub. Date: Oct. 2, 2025

(54) SYMPATHETIC NERVE ACTIVATION AGENT AND SYMPATHETIC NERVE ACTIVATION COMPOSITION

(71) Applicant: MARUZEN PHARMACEUTICALS CO., LTD., Hiroshima (JP)

(72) Inventor: Hiroyuki KAYAKI, Hiroshima (JP)

(21) Appl. No.: 18/864,243

(22) PCT Filed: Apr. 17, 2023
(86) PCT No.: PCT/JP2023/015382

§ 371 Date: Nov. 8, 2024

(30) Foreign Application Priority Data
May 13, 2022 (JP) 2022-079685

(51) Int. Cl. A61K 31/192; A61K 9/20; A61K 9/48; A61P 25/00

(52) U.S. Cl. CPC A61K 31/192; A61K 9/2018; A61K 9/2063; A61K 9/4825; A61K 9/4858; A61K 9/4866; A61P 25/00

(57) ABSTRACT

A sympathetic nerve activation agent includes a compound represented by Structural Formula (1).

### TECHNICAL FIELD

The present invention relates to sympathetic nerve activation agents and sympathetic nerve activation compositions.

### BACKGROUND ART

In recent years, stress has become a major problem. Stimuli externally applied to the mind and body are called stressors. Physical stressors, such as weather, temperature change, noise, and the like, chemical stressors, such as drugs, chemical substances that harm the body, and the like, and psychological and social stressors, such as human relationships, work-related problems, household problems, and the like, are known.

In order to maintain the internal environment constant against external stimuli, the system known as a autonomic nervous system is in a living body. The autonomic nerves include sympathetic nerves and parasympathetic nerves.

As the sympathetic nerves are stimulated, the sympathetic nerves act to increase heart rate, constrict blood vessels, lower gastrointestinal functions, or the like. The activation of the sympathetic nerves has expected effects of aiding clear thinking, reducing drowsiness, and maintaining concentration.

As a substance for activating sympathetic nerves, cyclo-propyl-phenylalanine, which is a cyclic dipeptide, has been reported (see, for example, Patent Document 1).

However, there is still a strong demand for a novel material that has an effect on sympathetic activation, and is highly safe, and therefore can be widely used as a substance for food and drink products, pharmaceutical products, research reagents, and the like.

Note that, the name of the compound represented by Structural Formula (1) below is 3-(4-hydroxy-3-methoxyphenyl) propionic acid. It has been known that the compound represented by Structural Formula (1) below can be detected when a certain type of lactic acid bacterium is cultured in a medium including a specific substance (see, for example, Patent Document 2).

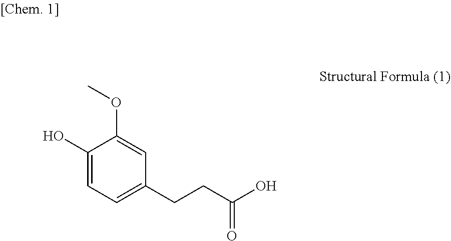

The compound represented by Structural Formula (1) is an active ingredient of a dipeptidyl peptidase IV activity inhibitor, and is known to be formulated in drink and food products for inhibiting dipeptidyl peptidase IV activity (see, for example, Patent Document 3).

However, it has not been known that the compound represented by Structural Formula (1) has a sympathetic activation effect.

### CITATION LIST

### Patent Documents

Patent Document 1: Japanese Unexamined Patent Application Publication No. 2022-048337
Patent Document 2: Japanese Unexamined Patent Application Publication No. 2014-003929
Patent Document 3: Japanese Unexamined Patent Application Publication No. 2020-055887

### SUMMARY OF INVENTION

### Technical Problem

The present inventors aim to solve the various problems existing in the related art, and achieve the following object. Specifically, the object of the present invention is to provide a sympathetic nerve activation agent and a sympathetic nerve activation composition, which have an excellent sympathetic activation effect and are highly safe.

### Solution to Problem

The present inventors have conducted research to solve the above problems. As a result, the present inventors have reached the insight such that a compound represented by Structural Formula (1) below has an excellent sympathetic activation effect, is highly safe, and is effective for use associated with sympathetic activation.

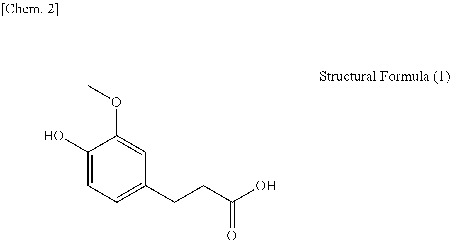

The present invention is based on the above insight of the present inventors, and the means for solving the above problems are as follows.

<1> A sympathetic nerve activation agent including a compound represented by Structural Formula (1) below

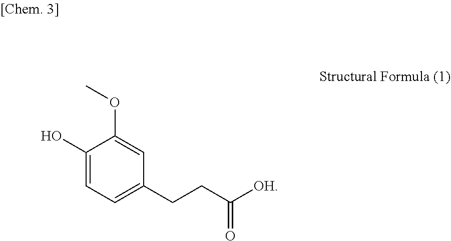

<2> The sympathetic nerve activation agent according to <1>, in which the sympathetic nerve is a sympathetic nerve innervating at least one selected from the group consisting of white adipose tissue, brown adipose tissue, adrenal glands, and liver.

<3> A sympathetic nerve activation composition including the sympathetic nerve activation agent according to <1> or <2>.

### Effects of Invention

According to the sympathetic nerve activation agent and sympathetic nerve activation composition of the present invention, the various problems existing in the related art can be solved, the above object can be achieved, and a sympathetic nerve activation agent and a sympathetic nerve activation composition, which have an excellent sympathetic activation effect and are highly safe, can be provided.

### BRIEF DESCRIPTION OF THE DRAWINGS

FIG. 1 is a diagram illustrating a change in neural activity of distal branches of the sympathetic nerve for epididymal white adipose tissue in Test Example 1.

FIG. 2 is a diagram illustrating a change in neural activity of distal branches of the sympathetic nerve for interscapular brown adipose tissue in Test Example 1.

FIG. 3 is a diagram illustrating a change in neural activity of distal branches of the sympathetic nerve for the adrenal gland in Test Example 1.

FIG. 4 is a diagram illustrating a change in neural activity of distal branches of the sympathetic nerve for the liver in Test Example 1.

### DESCRIPTION OF EMBODIMENTS

### (Sympathetic Nerve Activation Agent)

The sympathetic nerve activation agent of the present invention includes a compound represented by Structural Formula (1) below as an active ingredient, and may further include other ingredients as necessary.

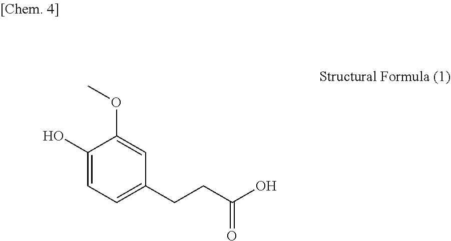

In the present specification, the sympathetic activation means that an activity level of sympathetic nerves is increased compared with a state before administering the sympathetic nerve activation agent.

The sympathetic nerve activation agent preferably activates the sympathetic nerve innervating at least one selected from the group consisting of white adipose tissue, brown adipose tissue, the adrenal glands, and the liver.

The fact that the compound represented by Structural Formula (1) has a sympathetic activation effect and is effective as a sympathetic nerve activation agent has not been known at all in the related art and is a novel insight of the present inventors.

### <Compound Represented by Structural Formula (1)>

The name of the compound represented by Structural Formula (1) below is 3-(4-hydroxy-3-methoxyphenyl) propionic acid) (may be referred to as “HMPA” hereinafter).

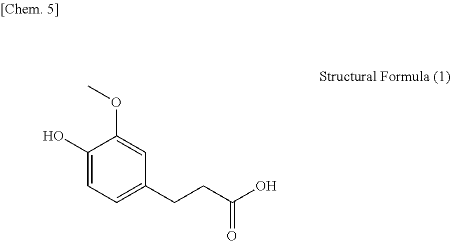

The compound represented by Structural Formula (1) is a known compound. As the compound represented by Structural Formula (1), a commercially available product may be used, or a compound extracted from plants of the like may be used.

An amount of the compound represented by Structural Formula (1) in the sympathetic nerve activation agent is not particularly limited, and may be appropriately selected.

The sympathetic nerve activation agent may be composed solely of the compound represented by Structural Formula (1), or may be obtained by formulating the compound represented by Structural Formula (1) as a preparation.

### <Other Ingredients>

The above other ingredients are not particularly limited, as long as the effects obtainable by the present invention are not adversely affected. The other ingredients are appropriately selected according to a usage form of the sympathetic nerve activation agent. Examples of the other ingredients include excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, alcohols, powder components, colorants, aqueous components, water, skin nutrients, and the like. The above other ingredients may be used alone or in combination.

An amount of the other ingredients in the sympathetic nerve activation agent is not particularly limited, and may be appropriately selected.

The sympathetic nerve activation agent can be formulated into a desired dosage form, such as powders, granules, tablets, liquids, or the like, using a pharmaceutically acceptable carrier, such as dextrin, cyclodextrin, or the like, or any other auxiliary agents according to a common method. As the auxiliary agents, for example, excipients, binders, disintegrating agents, lubricants, stabilizers, flavoring agents, or the like can be used.

The sympathetic nerve activation agent can be used by being blended in another composition (e.g., the below-described sympathetic nerve activation composition), and can be also used as a parenteral administration agent, such as injection, drip, suppository, and the like, or an ointment, an eye drop, a fluid preparation for external application, a patch, or the like.

Note that, the sympathetic nerve activation agent may use, as necessary, another component having a sympathetic activation effect blended together with the compound represented by Structural Formula (1) as active ingredients.

### <Use>

Use of the sympathetic nerve activation agent is not particularly limited, and may be appropriately selected. Examples of the use include a variety of use, such as pharmaceutical products, quasi-drugs, food and drink products, and the like.

Since the sympathetic nerve activation agent has an excellent sympathetic activation effect and is highly safe, the sympathetic nerve activation agent can be suitably used, for example, as an active ingredient of the below-described sympathetic nerve activation composition.

The sympathetic nerve activation agent is suitably applied to humans, but can be applied to animals other than humans (e.g., mice, rats, hamsters, dogs, cats, cows, pigs, monkeys, etc.) as long as the functions and effects of the sympathetic nerve activation agent can be exhibited.

The sympathetic nerve activation agent is suitably used for a subject whose activity level of the sympathetic nerves is lowered.

Usage of the sympathetic nerve activation agent is not particularly limited, and may be appropriately selected. Examples of the usage include oral administration, parenteral administration, external application, and the like.

A dosage form of the sympathetic nerve activation agent is not particularly limited, and may be appropriately selected from dosage forms known in the related art according to the intended purpose.

A production method for the sympathetic nerve activation agent in any dosage form is not particularly limited, and may be appropriately selected from methods known in the related art.

An administration method, an administration dose, an administration site, an administration period, an administration interval, and the like of the sympathetic nerve activation agent are not particularly limited, and may be appropriately selected.

For example, as an example of the administration dose of the sympathetic nerve activation agent in a human, the administration dose for a body weight of 60 kg per day, which is converted into the amount of the compound represented by Structural Formula (1), is from 0.5 mg to 5,000 mg, or the like.

Moreover, as an example of the administration dose of the sympathetic nerve activation agent in a human for enhancing the activity of the sympathetic nerve innervating white adipose tissue, the administration dose for a body weight of 60 kg per day, which is converted into the amount of the compound represented by Structural Formula (1), is from 50 mg to 5,000 mg, or the like.

Moreover, as an example of the administration dose of the sympathetic nerve activation agent in a human for enhancing the activity of the sympathetic nerve innervating brown adipose tissue, the administration dose for a body weight of 60 kg per day, which is converted into the amount of the compound represented by Structural Formula (1), is from 5 mg to 5,000 mg, or the like.

Moreover, as an example of the administration dose of the sympathetic nerve activation agent in a human for enhancing the activity of the sympathetic nerve innervating the adrenal glands, the administration dose for a body weight of 60 kg per day, which is converted into the amount of the compound represented by Structural Formula (1), is from 0.5 mg to 5,000 mg, or the like.

Moreover, as an example of the administration dose of the sympathetic nerve activation agent in a human for enhancing the activity of the sympathetic nerve innervating the liver, the administration dose for a body weight of 60 kg per day, which is converted into the amount of the compound represented by Structural Formula (1), is from 5 mg to 5,000 mg, or the like.

Moreover, the sympathetic nerve activation agent of the present invention can be also used as a reagent for research associated with an action mechanism of the sympathetic activation effect.

### (Sympathetic Nerve Activation Composition)

The sympathetic nerve activation composition of the present invention includes the sympathetic nerve activation agent of the present invention, and may further include other ingredients.

### <Sympathetic Nerve Activation Agent>

The sympathetic nerve activation agent is the above sympathetic nerve activation agent of the present invention.

An amount of the sympathetic nerve activation agent in the sympathetic nerve activation composition is not particularly limited, and may be appropriately adjusted according to a usage form of the sympathetic nerve activation composition or the like. The amount of the sympathetic nerve activation agent, which is converted into the amount of the compound represented by Structural Formula (1), is preferably from 0.0001% by mass to 20% by mass, and more preferably from 0.0001% by mass to 10% by mass. The sympathetic nerve activation composition may be composed solely of the sympathetic nerve activation agent.

### <Other Ingredients>

Other ingredients in the sympathetic nerve activation composition are not particularly limited, and may be appropriately selected according to a usage form of the sympathetic nerve activation composition. Examples of the above other ingredients include the same ingredients as the other ingredients described in the above section of the sympathetic nerve activation agent. Such ingredients may be used alone or in combination.

An amount of the other ingredients in the sympathetic nerve activation composition is not particularly limited, and may be appropriately selected.

### Embodiments

Embodiments of the sympathetic nerve activation composition are not particularly limited, and may be appropriately selected. Examples of the embodiments include pharmaceutical products, quasi-drugs, drink and food products, and the like.

The sympathetic nerve activation composition can be used daily, and can extremely effectively exhibit a variety of pharmacological activity including a sympathetic activation effect due to the action of the compound represented by Structural Formula (1) serving as an active ingredient.

The sympathetic nerve activation composition is suitably applied to humans, but can be applied to animals other than humans (e.g., mice, rats, hamsters, dogs, cats, cows, pigs, monkeys, etc.) as long as the functions and effects of the sympathetic nerve activation composition can be exhibited.

The sympathetic nerve activation composition is preferably used for a subject whose activity level of the sympathetic nerves is lower than a normal level.

Usage of the sympathetic nerve activation composition is not particularly limited, and may be appropriately selected. Examples of the usage include oral administration, parenteral administration, external application, and the like. The usage is preferably oral administration.

Examples of the composition for oral administration include oral preparations, food and drink products, and the like. In the present specification, the food and drink products refer to products that have little risk for harming human health and can be consumed in ordinary social life through oral administration or gastrointestinal administration. The food and drink products are not limited to categories according to the administrative classification, such as foods, pharmaceutical products, and quasi-drugs. Accordingly, the food and drink products include a wide range of food and drink products including general food products, health foods (functional food and drink products), health-promoting food products (food for specified health uses, food with nutrient function claims, and food with functional claims), quasi-drugs, pharmaceutical products, etc., which are for oral intake.

A type of the composition for the oral administration is not particularly limited, and may be appropriately selected. Examples thereof include: beverages such as tea beverages, soft drinks, carbonated beverages, nutritional beverages, fruit-based beverages, lactic beverages, alcoholic beverages, coffee beverages, coffee-containing soft drinks, and the like (including concentrates of the foregoing beverages and powders for preparing the foregoing beverages); frozen desserts such as ice cream, sherbet, shaved ice, and the like; noodles, such as buckwheat noodles, udon noodles, starch noodles, Gyoza skins, Shumai skins, Chinese noodles, instant noodles, and the like; confectionary, such as hard candies, candies, chewing gums, chocolate, tablets, snacks, biscuits, jelly, jams, creams, sweet pastries, bread, and the like; fishery products, such as crabs, salmon, clams, tuna, sardine, shrimp, bonito, mackerel, whale, oysters, Pacific saury, squid, Anadara broughtonii, scallops, abalone, sea urchin, salmon roe, Sulculus diversicolor supertexta, and the like; fish or meat processed foods, such as fish paste, ham, sausages, and the like; dairy products, such as processed milk, fermented milk, and the like; fat or oil, or fat or oil processed food products, such as salad oil, tempura oil, margarines, mayonnaise, shortening, whipping creams, dressing, and the like; seasonings, such as sauces and dipping sauces; retort pouch-packaged foods, such as curry, stew, Oyakodon, gruel, rice soups, Chukadon, a bowl of rice with breaded pork cutlets, a bowl of rice with tempura, Unadon, Hayashi rice, Oden, Mapo tofu, a bowl of rice with beef, Bolognese sauces, egg soup, omelette made with rice, Gyoza, Shumai, hamburgers, meat balls, and the like; daily dishes, such as salads and pickles; various forms of health, cosmetic, or nutritional supplementary foods; pharmaceutical products and quasi-drugs, such as tablets, powders, capsules, granules, extracts, syrups, drink preparations, lozenges, mouthwash, and the like; oral fresheners used inside the mouth, such as mouth freshener and breath freshener; toothpaste; and the like.

A production method for the sympathetic nerve activation composition is not particularly limited and may be appropriately selected according to a usage form of the sympathetic nerve activation composition.

A usage dose, a usage site, a usage period, a usage interval and the like of the sympathetic nerve activation composition are not particularly limited, and may be appropriately selected.

For example, as an example of the usage dose of the sympathetic nerve activation composition in a human, the usage dose for a body weight of 60 kg per day, which is converted into the amount of the compound represented by Structural Formula (1), is from 0.5 mg to 5,000 mg, or the like.

Moreover, as an example of the usage dose of the sympathetic nerve activation composition in a human for enhancing the activity of the sympathetic nerve innervating white adipose tissue, the usage dose for a body weight of 60 kg per day, which is converted into the amount of the compound represented by Structural Formula (1), is from 50 mg to 5,000 mg, or the like.

Moreover, as an example of the usage dose of the sympathetic nerve activation composition in a human for enhancing the activity of the sympathetic nerve innervating brown adipose tissue, the usage dose for a body weight of 60 kg per day, which is converted into the amount of the compound represented by Structural Formula (1), is from 5 mg to 5,000 mg, or the like.

Moreover, as an example of the usage dose of the sympathetic nerve activation composition in a human for enhancing the activity of the sympathetic nerve innervating the adrenal glands, the usage dose for a body weight of 60 kg per day, which is converted into the amount of the compound represented by Structural Formula (1), is from 0.5 mg to 5,000 mg, or the like.

Moreover, as an example of the usage dose of the sympathetic nerve activation composition in a human for enhancing the activity of the sympathetic nerve innervating the liver, the usage dose for a body weight of 60 kg per day, which is converted into the amount of the compound represented by Structural Formula (1), is from 5 mg to 5,000 mg, or the like.

As described above, the sympathetic nerve activation agent and sympathetic nerve activation composition of the present invention have an excellent sympathetic activation effect.

Accordingly, the present invention also relates to a method of activating sympathetic nerves including administration of at least one selected from the group consisting of the sympathetic nerve activation agent and the sympathetic nerve activation composition to a subject.

In addition, as the activity of the sympathetic nerve innervating the white adipose tissue is activated, lipase in the white adipose tissue is activated via β3-adrenergic receptors to facilitate lipolysis that decomposes the accumulated neutral fat into a fatty acid and glycerin, thereby achieving a diet effect of reducing the amount of the accumulated fat.

As the activity level of the sympathetic nerve innervating the brown adipose tissue is increased, uncoupling protein-1 (UCP-1) in mitochondria of the brown adipose tissue is activated via β3-adrenergic receptors to facilitate heat production, thereby elevating a body temperature. The heat production of the above tissue uses fatty acid, and therefore energy consumption is increased, thereby achieving a diet effect. In addition, it has been reported that the physical and mental work load and the accuracy of the work increase when the temperature of the human body is high, thereby enhancing working performance.

As the activity level of the sympathetic nerve innervating the adrenal glands is increased, the release of adrenaline and noradrenaline from the adrenal medulla into the blood (endocrine secretion) is facilitated. As a result, the response to the released adrenaline and noradrenaline in organs and tissues of the body is determined by a type of adrenergic receptors present in respective sites. As the sympathetic nerve of the adrenal glands is facilitated, adrenaline and noradrenaline are generally secreted to enhance functions, energy mobilization and activity level of the heart.

As the activity level of the sympathetic nerve innervating the liver is increased, the parasympathetic nerve innervating the liver is suppressed. As a result, glycogenolysis enzymes are activated to release glucose generated by the glycogenolysis, in addition to facilitation of gluconeogenesis in the liver, thereby increasing an amount of glucose supplied to the entire body.

Accordingly, the present invention also relates to a lipolysis promoter, a fat accumulation inhibitor, or an activity promoter for increasing an activity level of a subject, which all include the compound represented by the structural formula (1). Moreover, the present invention also relates to a lipolysis promoting composition including the lipolysis promoter, a fat accumulation inhibiting composition including the fat accumulation inhibitor, or an activity promoting composition including the activity promoter. Further, the present invention also relates to a lipolysis promoting method including administration of at least one selected from the group consisting of the lipolysis promoter and the lipolysis promoting composition to a subject, a fat accumulation inhibiting method including administration of at least one selected from the group consisting of the fat accumulation inhibitor and the fat accumulation inhibiting composition to a subject, or an activity promoting method of increasing an activity level of a subject, which includes administration of at least one selected from the group consisting of the activity promoter and the activity promoting composition to a subject.

### EXAMPLES

Test Example and Formulation Examples of the present invention will be described hereinafter, but the present invention is not limited to Text Example and Formulation Examples below.

### Test Example 1

The effect of the intragastric administration of the compound represented by Structural Formula (1) on the activity of the sympathetic nerve innervating the white adipose tissue, the brown adipose tissue, the adrenal gland, or the liver was tested in the following manner.

### <Measurement>

For the experiment, male Whistar rats (approximately 9 weeks old) each having a body weight of approximately 300 g were kept in a 24° C. thermostatic animal room for 1 week or longer with a 12-hour light-dark cycle (lighting from 8:00 to 20:00). On the day of the experiment, the rats were fasted for 3 hours, followed by anesthetizing with urethane. A cannula for intragastric administration was inserted into each of the rats, and then distal branches of the sympathetic nerve innervating the right epididymal white adipose tissue, the interscapular brown adipose tissue, the left adrenal gland, or the liver were lifted with a silver electrode, and the electrical activity thereof was measured.

When the measurement value stabilized (at approximately 13:00), an aqueous solution of the compound represented by Structural Formula (1) (produced by Tokyo Chemical Industry Co., Ltd.) was intragastrically administered using the cannula at a dose of 15 mg/0.5 mL/300 g of body weight, 1.5 mg/0.5 mL/300 g of body weight, 0.15 mg/0.5 mL/300 g of body weight, or 0.015 mg/0.5 mL/300 g of body weight, and the change in the neural activity of the distal branches of the above sympathetic nerves was measured electrophysiologically for 60 minutes. In addition, as a control, water serving as a solvent was intragastrically administered using the cannula at a dose of 0.5 mL/300 g of body weight, and the change in the neural activity of the distal branches of the above sympathetic nerves was measured electrophysiologically for 60 minutes.

From the start of the operation to the end of the measurement, a tube was inserted into the trachea to secure an airway, and the body temperature (rectal temperature of the rat) was maintained at 37.0±0.5° C. with a warming device.

The data of the neural activity was analyzed as the mean value of the firing rate per 5 seconds (pulse/5 s) every 5 minutes, and represented by a percentage relative to the mean value (a value at 0 minutes) at 5 minutes before the onset of stimulation being determined as 100%.

### <Results>

### White Adipose Tissue

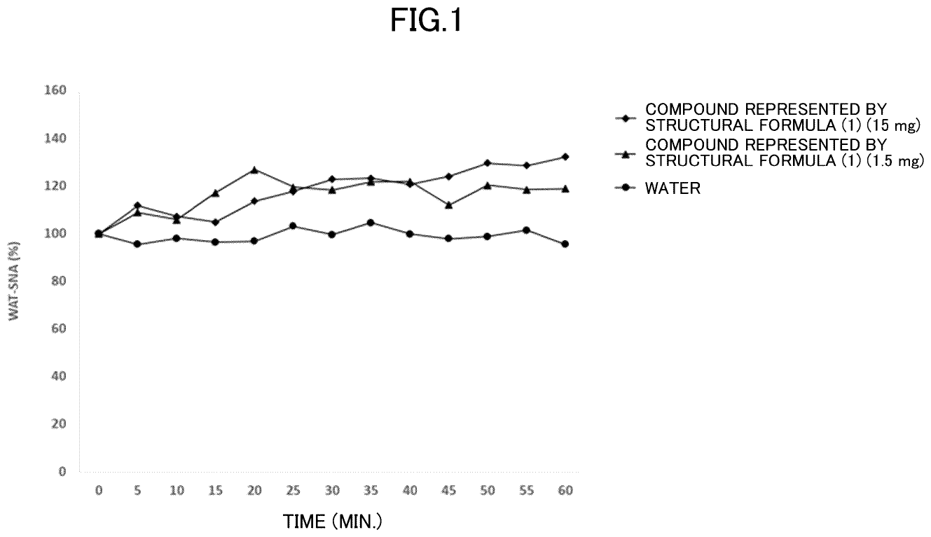

FIG. 1 illustrates, as a percentage with respect to the mean value (a value at 0 minutes) at 5 minutes before the onset of stimulation being determined as 100%, the result of the change in the neural activity of distal branches of the sympathetic nerves for the epididymal white adipose tissue (white adipose tissue sympathetic nerve activity, WAT-SNA) when the aqueous solution including 15 mg or 1.5 mg of the compound represented by Structural Formula (1) was intragastrically administered at a dose of 0.5 mL/300 g of body weight, or water serving as the solvent was intragastrically administered at a dose of 0.5 mL/300 g of body weight.

As presented in FIG. 1, it was made clear that the group to which the aqueous solution including 15 mg of the compound represented by Structural Formula (1) was administered at the dose of 0.5 mL/300 g of body weight (“♦” in FIG. 1) and the group to which the aqueous solution including 1.5 mg of the compound represented by Structural Formula (1) was administered at the dose of 0.5 mL/300 g of body weight (“▴” in FIG. 1) had the WAT-SNA values higher than the WAT-SNA value of the group to which water serving as the solvent was administered at the dose of 0.5 mL/300 g of body weight (“●” in FIG. 1). It was found from the above results that the compound represented by Structural Formula (1) had the effect of activating the activity of the sympathetic nerve innervating the white adipose tissue.

As the activity level of the sympathetic nerve innervating the white adipose tissue is increased, lipase in the white adipose tissue is activated via β3-adrenergic receptors to facilitate lipolysis that decomposes the accumulated neutral fat into a fatty acid and glycerin, thereby achieving a diet effect of reducing the amount of the accumulated fat.

### Brown Adipose Tissue

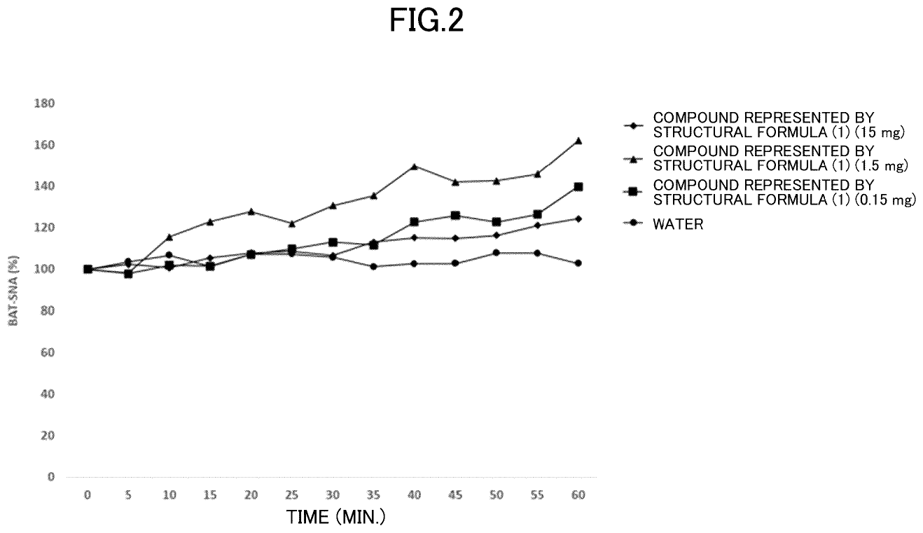

FIG. 2 illustrates, as a percentage with respect to the mean value (a value at 0 minutes) at 5 minutes before the onset of stimulation being determined as 100%, the result of the change in the neural activity of distal branches of the sympathetic nerves for interscapular brown adipose tissue (brown adipose tissue sympathetic nerve activity, BAT-SNA) when the aqueous solution including 15 mg, 1.5 mg, or 0.15 mg of the compound represented by Structural Formula (1) was intragastrically administered at a dose of 0.5 mL/300 g of body weight, or water serving as the solvent was intragastrically administered at a dose of 0.5 mL/300 g of body weight.

As presented in FIG. 2, it was made clear that the group to which the aqueous solution including 15 mg of the compound represented by Structural Formula (1) was administered at the dose of 0.5 mL/300 g of body weight (“♦” in FIG. 2), the group to which the aqueous solution including 1.5 mg of the compound represented by Structural Formula (1) was administered at the dose of 0.5 mL/300 g of body weight (“▴” in FIG. 2), and the group to which the aqueous solution including 0.15 mg of the compound represented by Structural Formula (1) was administered at the dose of 0.5 mL/300 g of body weight (“▪” in FIG. 2) had the BAT-SNA values higher than the BAT-SNA value of the group to which water serving as the solvent was administered at the dose of 0.5 mL/300 g of body weight (“●” in FIG. 2). It was found from the above results that the compound represented by Structural Formula (1) had the effect of activating the activity of the sympathetic nerve innervating the brown adipose tissue.

As the activity level of the sympathetic nerves innervating the brown adipose tissue is increased, uncoupling protein-1 (UCP-1) in mitochondria of the brown adipose tissue is activated via β3-adrenergic receptors to facilitate heat production, thereby elevating a body temperature. The heat production of the above tissue uses fatty acid, and therefore energy consumption is increased, thereby achieving a diet effect. In addition, it has been reported that the physical and mental work load and the accuracy of the work increase when the temperature of the human body is high, thereby enhancing working performance.

### Adrenal Gland

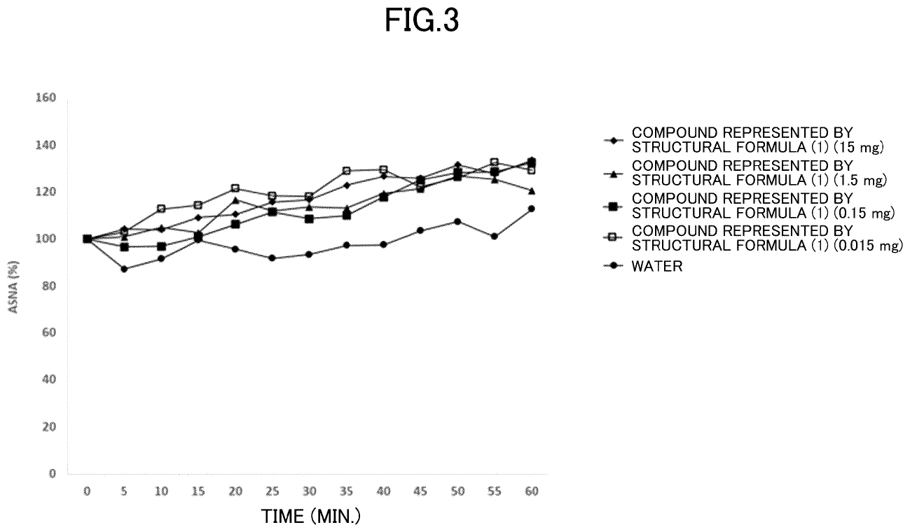

FIG. 3 illustrates, as a percentage with respect to the mean value (a value at 0 minutes) at 5 minutes before the onset of stimulation being determined as 100%, the result of the change in the neural activity of distal branches of the sympathetic nerves for the adrenal gland (adrenal sympathetic nerve activity, ASNA) when the aqueous solution including 15 mg, 1.5 mg, 0.15 mg, or 0.015 mg of the compound represented by Structural Formula (1) was intragastrically administered at a dose of 0.5 mL/300 g of body weight, or water serving as the solvent was intragastrically administered at a dose of 0.5 mL/300 g of body weight.

As presented in FIG. 3, it was made clear that the group to which the aqueous solution including 15 mg of the compound represented by Structural Formula (1) was administered at the dose of 0.5 mL/300 g of body weight (“♦” in FIG. 3), the group to which the aqueous solution including 1.5 mg of the compound represented by Structural Formula (1) was administered at the dose of 0.5 mL/300 g of body weight (“▴” in FIG. 3), the group to which the aqueous solution including 0.15 mg of the compound represented by Structural Formula (1) was administered at the dose of 0.5 mL/300 g of body weight (“▪” in FIG. 3), and the group to which the aqueous solution including 0.015 mg of the compound represented by Structural Formula (1) was administered at the dose of 0.5 mL/300 g of body weight (“□” in FIG. 3) had the ASNA values higher than the ASNA value of the group to which water serving as the solvent was administered at the dose of 0.5 mL/300 g of body weight (“●” in FIG. 3). It was found from the above results that the compound represented by Structural Formula (1) had the effect of activating the activity of the sympathetic nerve innervating the adrenal gland.

As the activity level of the sympathetic nerve innervating the adrenal glands is increased, the release of adrenaline and noradrenaline from the adrenal medulla into the blood (endocrine secretion) is facilitated. As a result, the response to the released adrenaline and noradrenaline in organs and tissues of the body is determined by a type of adrenergic receptors present in respective sites. As the sympathetic nerve of the adrenal glands is facilitated, adrenaline and noradrenaline are generally secreted to enhance functions, energy mobilization and activity level of the heart. Therefore, the administration of the compound represented by Structural Formula (1) can facilitate secretion of adrenaline and noradrenaline from the adrenal medulla, thereby achieving effects of enhancing functions, energy mobilization and activity level of the heart.

### Liver

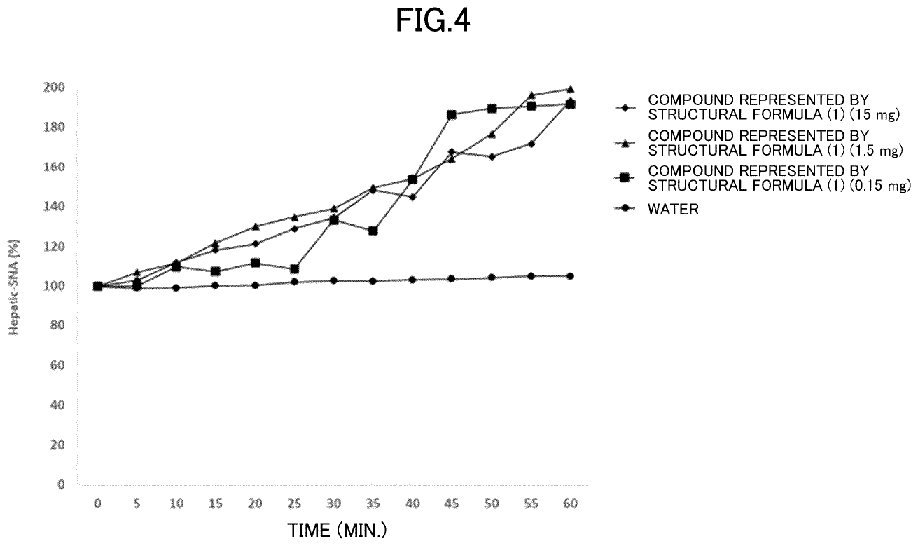

FIG. 4 illustrates, as a percentage with respect to the mean value (a value at 0 minutes) at 5 minutes before the onset of stimulation being determined as 100%, the result of the change in the neural activity of distal branches of the sympathetic nerves for the liver (hepatic sympathetic nerve activity, Hepatic-SNA) when the aqueous solution including 15 mg, 1.5 mg, or 0.15 mg of the compound represented by Structural Formula (1) was intragastrically administered at a dose of 0.5 mL/300 g of body weight, or water serving as the solvent was intragastrically administered at a dose of 0.5 mL/300 g of body weight.

As presented in FIG. 4, it was made clear that the group to which the aqueous solution including 15 mg of the compound represented by Structural Formula (1) was administered at the dose of 0.5 mL/300 g of body weight (“♦” in FIG. 4), the group to which the aqueous solution including 1.5 mg of the compound represented by Structural Formula (1) was administered at the dose of 0.5 mL/300 g of body weight (“▴” in FIG. 4), and the group to which the aqueous solution including 0.15 mg of the compound represented by Structural Formula (1) was administered at the dose of 0.5 mL/300 g of body weight (“▪” in FIG. 4) had the Hepatic-SNA values higher than the Hepatic-SNA value of the group to which water serving as the solvent was administered at the dose of 0.5 mL/300 g of body weight (“●” in FIG. 4). It was found from the above results that the compound represented by Structural Formula (1) had the effect of activating the activity of the sympathetic nerve innervating the liver.

As the activity level of the sympathetic nerve innervating the liver is increased, the parasympathetic nerve innervating the liver is suppressed. As a result, glycogenolysis enzymes are activated to release glucose generated by the glycogenolysis, in addition to facilitation of gluconeogenesis in the liver, thereby increasing an amount of glucose supplied to the entire body.

### Formulation Example 1

Tablets having the following formulation were prepared according to a common practice.
The compound represented by Structural Formula (1): 5.0 mg Dolomite: 83.4 mg (including 20% of calcium and 10% of magnesium)
Casein phosphopeptide: 16.7 mg
Vitamin C: 33.4 mg
Maltitol: 136.8 mg
Collagen: 12.7 mg
Sucrose fatty acid ester: 12.0 mg

### Formulation Example 2

An oral liquid preparation having the following formulation was prepared according to a common practice.

### <Formulation for One Ampule (100 mL Per Ampule)>

The compound represented by Structural Formula (1): 0.3% by mass
Sorbitol: 12.0% by mass
Sodium benzoate: 0.1% by mass
Fragrance: 1.0% by mass
Calcium sulfate: 0.5% by mass
Purified water: balance

### Formulation Example 3

A coffee drink having the following formulation was prepared according to a common practice.

The compound represented by Structural Formula (1): 0.1% by mass
Coffee extract: 40.0% by mass (L (brightness)=20, Brix=3)
Maltitol: 2.0% by mass
Fragrance: appropriate amount
Water: balance

### Formulation Example 4

Capsules having the following formulation were prepared according to common practice. As capsules, hard gelatin No. 1 capsules were used.

### <Composition in 1 Capsule (200 mg Per Capsule)>

The compound represented by Structural Formula (1): 30.0 mg
Corn starch: 70.0 mg
Lactose: 80.0 mg
Calcium lactate: 10.0 mg
Hydroxypropyl cellulose (HPC-L): 10.0 mg

The present application claims priority based on Japanese Patent Application No. 2022-079685, filed on May 13, 2022, the entire content of which are incorporated herein by reference.

1. A sympathetic nerve activation agent comprising:
a compound represented by Structural Formula (1) below,

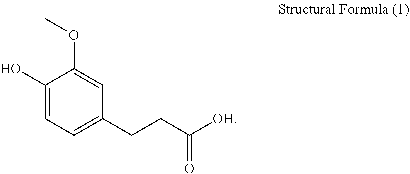

2. The sympathetic nerve activation agent according to claim 1,
wherein the sympathetic nerve is a sympathetic nerve innervating at least one selected from a group consisting of white adipose tissue, brown adipose tissue, adrenal glands, and liver.

3. A sympathetic nerve activation composition, comprising:
the sympathetic nerve activation agent according to claim 1; and
at least one selected from the group consisting of excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, alcohols, powder components, colorants, aqueous components, water, skin nutrients, and auxiliary agents.

4. A sympathetic nerve activation composition, comprising:
the sympathetic nerve activation agent according to claim 2; and
at least one selected from the group consisting of excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, alcohols, powder components, colorants, aqueous components, water, skin nutrients, and auxiliary agents.
